(19) United States
(12) Patent Application Publication
(10) Pub. No.: US 2018/0344326 A1
(43) Pub. Date: Dec. 6, 2018

(54) METHOD FOR DESIGNING A PATIENT SPECIFIC INSTRUMENT FOR AN ORTHOPEDIC SURGERY

(71) Applicants: Chang Gung Memorial Hospital, Linkou, Taoyuan City (TW); National Taiwan University of Science and Technology, Taipei City (TW)

(72) Inventors: Yi-Sheng Chan, Taoyuan City (TW); Chi-Pin Hsu, Taoyuan City (TW); Shang-Chih Lin, New Taipei City (TW); Shih-Sheng Chang, Taoyuan City (TW)

(73) Assignees: Chang Gung Memorial Hospital, Linkou, Taoyuan City (TW); National Taiwan University of Science and Technology, Taipei City (TW)

(21) Appl. No.: 15/992,806

(22) Filed: May 30, 2018

(30) Foreign Application Priority Data
May 31, 2017 (TW) 106117817

(51) Int. Cl. A61B 17/15; A61B 17/17

(52) U.S. Cl. CPC A61B 17/154; A61B 17/1675; A61B 17/1764

(57) ABSTRACT

A method for designing an instrument for orthopedic surgery includes obtaining imaging information associated with a bone segment of a subject and an implant fastened to the bone segment, creating a bone segment model and an implant model based on the imaging information associated with the bone segment and the implant, and generating information regarding a shape of an instrument based on the bone segment model and the implant model. The instrument is to be disposed on the bone segment and the implant. The instrument is formed with a path to align a positioning component with a location of the bone segment which is not covered by the implant and at which the positioning component is to engage the bone segment.

### CROSS-REFERENCE TO RELATED APPLICATION

This application claims priority of Taiwanese Patent Application No. 106117817, filed on May 31, 2017.

### FIELD

The disclosure relates to a method for designing a patient specific instrument, more particularly to a method for designing a patient specific instrument for an orthopedic surgery.

### BACKGROUND

Tibial plateau fracture is a common issue involving the knee joint of a patient, and may typically be treated by performing a surgery known as orthopedic surgery. In the orthopedic surgery, a patient specific instrument may be used for allowing operations such as cutting or drilling of a bone to be done with more accuracy.

It is noted that after an initial surgery, in which implants such as a bone plate and a bone nail are implanted, if recovery of fractured part of the bone is incomplete and/or a fractured limb is not immobilized in an appropriate manner, a condition called malunion may occur, accompanied by complications such as a bone defect, a cyllum, gonyectyposis, and/or unstable knee joint.

When the complications from the malunion occur, an additional surgery may be required with the implants still in the patient's body. In this case, performing additional surgery may be more difficult due to the presence of the implants. For example, at a preoperative planning stage for the surgery and/or during the actual surgery, it may be required to obtain multiple X-ray images in order to determine the location of the implants.

### SUMMARY

Therefore, one object of the disclosure is to provide a method for designing a patient specific instrument for an orthopedic surgery.

According to one embodiment of the disclosure, the method is performed using a processor and includes:
obtaining imaging information associated with a bone segment of a subject and an implant fastened to a part of the bone segment;
creating a three-dimensional bone segment model for the bone segment and a three-dimensional implant model for the implant based on the imaging information associated with the bone segment and the implant; and
generating geometric information regarding a shape of a positioning surgical instrument based on the three-dimensional bone segment model and the three-dimensional implant model, the positioning surgical instrument to be disposed on the bone segment and the implant, the positioning surgical instrument being formed with at least one positioning path that guides passage of a positioning component therethrough and aligns the positioning component with a positioning location of the bone segment which is not covered by the implant and at which the positioning component is to engage the bone segment.

According to another aspect of this disclosure, there is disclosed a patient specific instrument for an orthopedic surgery. The patient specific instrument includes a base and a plurality of positioning parts. The base is shaped to be disposed on a bone segment of a subject and an implant fastened to a part of the bone segment. The positioning parts extend from the base. Each of the positioning parts is formed with a positioning slot that defines a positioning path that guides passage of a positioning component therethrough, and that aligns the positioning component with a positioning location of the bone segment which is not covered by the implant and at which the positioning component is to engage the bone segment.

### BRIEF DESCRIPTION OF THE DRAWINGS

Other features and advantages of the disclosure will become apparent in the following detailed description of the embodiments with reference to the accompanying drawings, of which:

FIG. 1 is a flow chart illustrating steps of a method for designing a patient specific instrument for an orthopedic surgery according to one embodiment of the disclosure;

FIG. 2 illustrates a first implant fastened on a bone segment of a subject;

FIG. 3 is a flow chart illustrating sub-steps for creating a three-dimensional bone segment model and a three-dimensional first implant model;

FIG. 4 illustrates the bone segment with the first implant, and a positioning surgical instrument to be disposed on the bone segment according to one embodiment of the disclosure;

FIG. 5 illustrates the positioning surgical instrument disposed on the bone segment, and a pair of positioning components for engaging the bone segment;

FIG. 6 illustrates the positioning components engaging the bone segment;

FIG. 7 illustrates a guiding surgical instrument to be disposed on the bone segment, with assistance of the positioning components according to one embodiment of the disclosure;

FIG. 8 illustrates the guiding surgical instrument disposed on the bone segment, and a pair of securing pins;

FIG. 9 illustrates a portion of the bone segment being cut, and a second implant to be disposed on the bone segment according to one embodiment of the disclosure;

FIG. 10 illustrates a first implant fastened on a bone segment of a subject, and a positioning surgical instrument and a guiding surgical instrument according to one embodiment of the disclosure;

FIG. 11 illustrates a first implant fastened on a bone segment of a subject, and a positioning surgical instrument and a guiding surgical instrument according to one embodiment of the disclosure; and

FIG. 12 illustrates a computer tomography image with beam-hardening effect.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

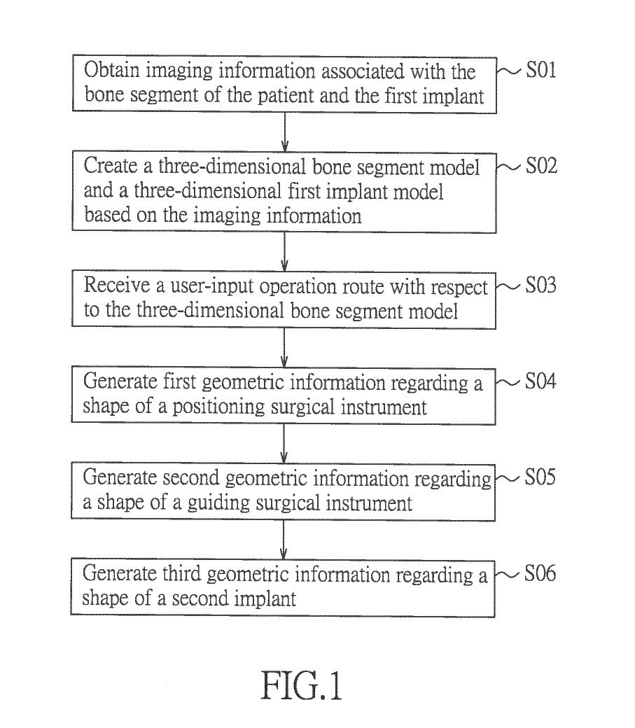

FIG. 1 is a flow chart illustrating a method for designing a patient specific instrument for an orthopedic surgery, according to one embodiment of the disclosure.

Steps of the method may be implemented using a computing device (not depicted in the drawings) that includes a processor which has computing capabilities and which is programmable to perform acts and algorithm in the following description.

In this embodiment, the method is implemented specifically for a subject, who may be a patient with a knee joint issue, such as tibial plateau fracture with depression, and has been subjected to a prior orthopedic surgery, but is not limited as such.

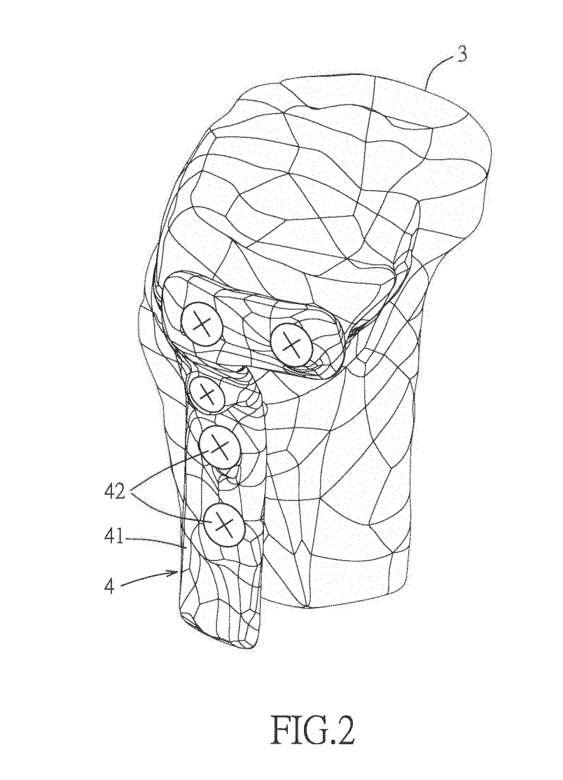

FIG. 2 illustrates a bone segment 3 of the patient, and a first implant 4 that is fastened to the bone segment 3 as a result of the prior orthopedic surgery. In this embodiment, the first implant 4 includes a first bone plate 41 and a plurality of first bone screws 42 put in the bone segment 3 respectively at first spots thereof for securing the first bone plate 41 on the bone segment 3.

Referring back to FIG. 1, in step S01, the processor obtains imaging information associated with the bone segment 3 of the patient and the first implant 4.

In this embodiment, the imaging information includes a plurality of computed tomography (CT) scan images of the bone segment 3 and the first implant 4. In other embodiments, the imaging information may include images captured by other image capturing techniques, such as images captured using X-ray or an optical scanning instrument.

In step S02, the processor creates a three-dimensional bone segment model which represents the bone segment 3 and a three-dimensional first implant model which represents the first implant 4 based on the imaging information.

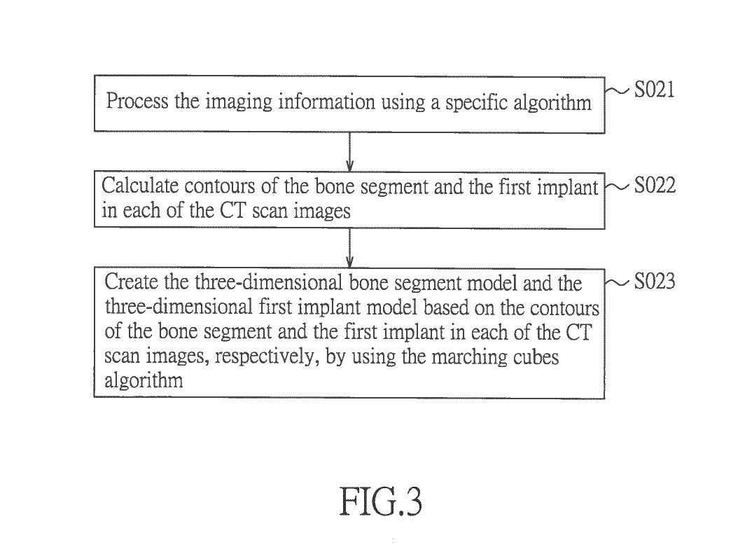

Specifically, step S02 may be implemented in a manner as depicted in FIG. 3.

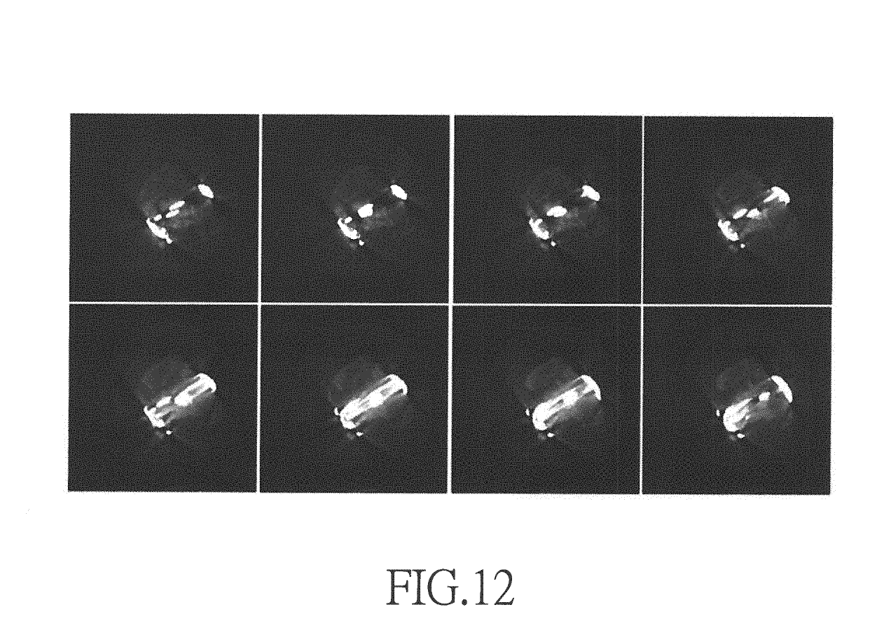

In sub-step S021, the processor processes the imaging information using a specific algorithm. In this embodiment, the specific algorithm may be one of interpolate correction, iterative correction and combines correction. This processing is done in order to reduce noise effects of an artifact resulting from beam hardening with respect to metal material of the first implant 4 (as seen in FIG. 12).

In sub-step S022, the processor calculates contours of the bone segment 3 and the first implant 4 in each of the CT scan images based on the imaging information thus processed using region growing by pixel aggregation.

Afterward, in sub-step S023, the processor creates the three-dimensional bone segment model and the three-dimensional first implant model based on the contours of the bone segment and the first implant in each of the CT scan images, respectively, by using the marching cubes algorithm. It is noted that the resultant three-dimensional bone segment model and the three-dimensional first implant model may then be processed separately or as a whole by the processor.

After the three-dimensional bone segment model and the three-dimensional first implant model are both created, the flow proceeds to step S03, in which the processor receives a user-input operation route with respect to the three-dimensional bone segment model indicating at least one position of a cut to be performed on the bone segment 3 during the orthopedic surgery.

Specifically, by inspecting the three-dimensional bone segment model and the three-dimensional first implant model, an operator (e.g., a surgeon) may determine an appropriate operation to be performed on the bone segment 3 (e.g., cutting, drilling, or a combination thereof) and a position of the bone segment 3 on which the operation is to be performed.

In this embodiment, a specific portion of the bone segment is determined to have depression, and it may be determined that the specific portion needs to be first cut off and then elevated to a new location. In order to successfully cut the specific portion, the surgeon needs to consider the presence of the first implant 4 with respect to the bone segment 3, and to plan the operation route accordingly.

With the operation route ready, in step S04, the processor generates first geometric information regarding a shape of a positioning surgical instrument, based on the three-dimensional bone segment model, the three-dimensional first implant model and the operation route. It is noted that, with the first geometric information, the positioning surgical instrument may be created physically using, for example, additive manufacturing or three-dimensional (3D) printing.

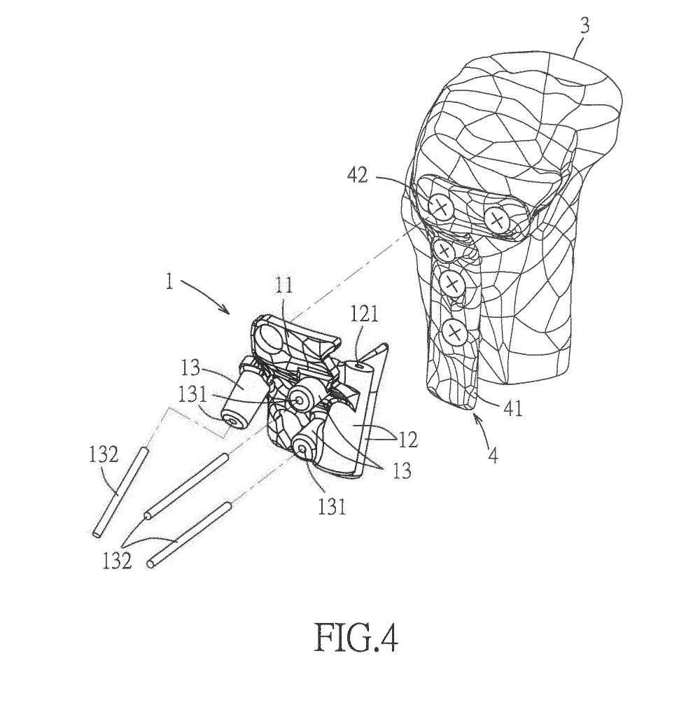

FIG. 4 illustrates an exemplary positioning surgical instrument 1 to be disposed on the bone segment 3 and the first implant 4. In this embodiment, the positioning surgical instrument 1 includes a base 11, a plurality of (e.g., two) positioning parts 12 extending from the base 11, and a plurality of (e.g., three) securing parts 13 extending from the base 11.

Each of the securing parts 13 is formed with a securing slot 131. The securing slot 131 defines a securing path that guides passage of a securing component 132 therethrough, and aligns the securing component 132 with a securing location of the bone segment 3 which is not covered by the first implant 4 and at which the securing component 132 is to engage the bone segment 3.

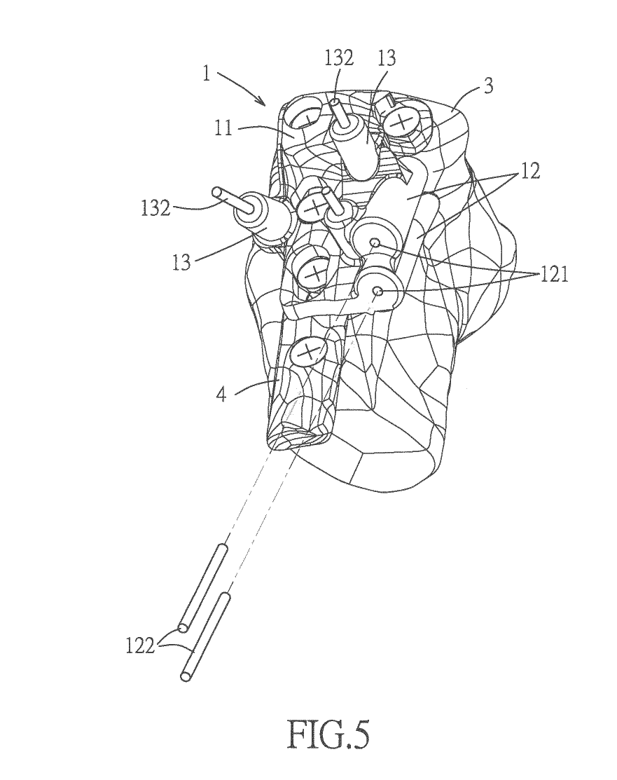

FIG. 5 illustrates the securing components 132 engaging the securing locations of the bone segment 3 so that the positioning surgical instrument 1 can be secured onto the bone segment 3 and the first implant 4. In this embodiment, the securing components 132 are embodied using pins.

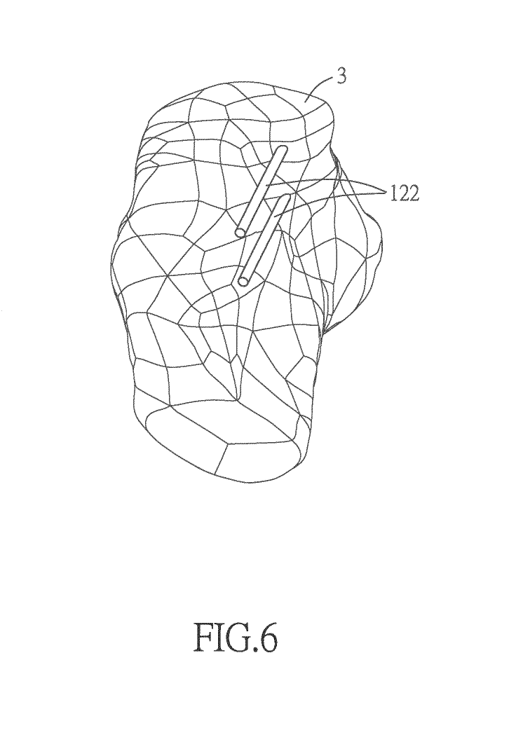

Each of the positioning parts 12 is formed with a first positioning slot 121. The first positioning slot 121 defines a first positioning path that guides passage of a positioning component 122 (see FIG. 6) therethrough, and aligns the positioning component 122 with a positioning location of the bone segment 3 which is not covered by the first implant 4 and at which the positioning component 122 is to engage the bone segment 3. The positioning component 122 is exemplified as but is not limited to a pin, and a function thereof will be discussed later.

Moreover, the first positioning path is formed on the positioning surgical instrument 1 to allow the positioning component 122 to engage the bone segment 3 at the positioning location which corresponds to the position of the cut indicated by the operation route. In this manner, a number of holes to be drilled on the bone segment 3 during the orthopedic surgery may be reduced, facilitating postoperative recovery.

As shown in FIG. 6, after the positioning components 122 are made to engage the bone segment 3, the first implant 4 (see FIG. 4), the positioning surgical instrument 1 (see FIG. 4) and the securing components 132 (see FIG. 4) may be removed from the bone segment 3.

In step S05, the processor generates second geometric information regarding a shape of a guiding surgical instrument based on the three-dimensional bone segment model and the user-input operation route. It is noted that, with the second geometric information, the guiding surgical instrument may be created physically using, for example, additive manufacturing or 3D printing.

The guiding surgical instrument is to be disposed on the bone segment 3 with the first implant 4 removed from the bone segment 3.

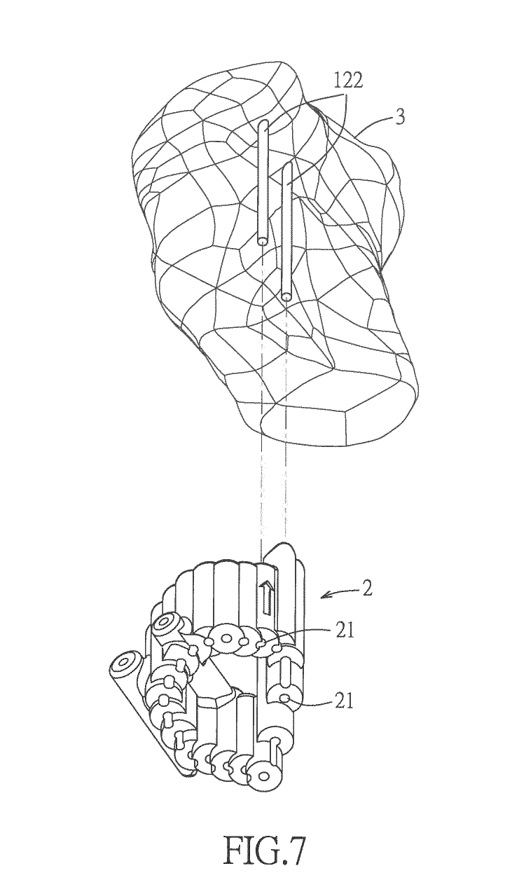

Referring to FIG. 7, an exemplary guiding surgical instrument 2 is formed with a plurality of (e.g., two) second positioning slots 21. Each of the positioning slots 21 defines a second positioning path that is configured to for insertion by a corresponding one of the positioning components 122, which has engaged the bone segment 3 at the positioning location. As an example, the guiding surgical instrument 2 is formed with two second positioning slots 21 in FIG. 7. Due to the presence and assistance of the positioning components 122, the guiding surgical instrument 2 may be easily positioned at a desired location with respect to the bone segment 3.

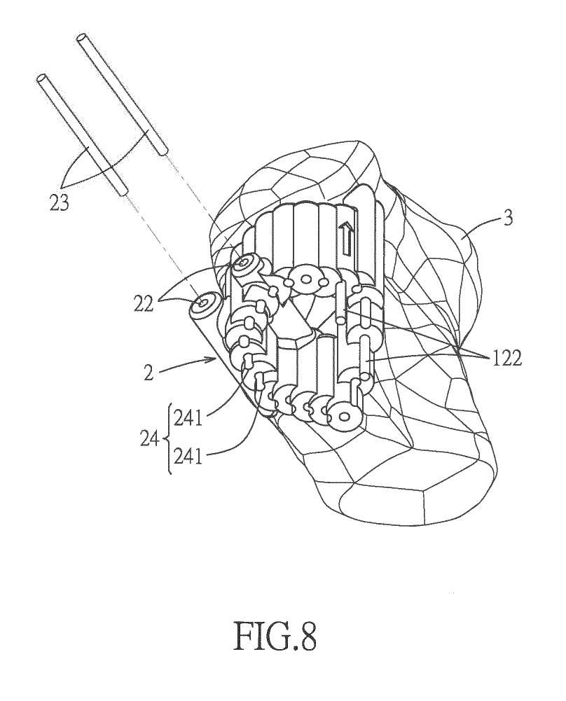

Further referring to FIG. 8, the guiding surgical instrument 2 is further formed with a plurality of (e.g., two) through holes 22 for allowing a plurality of (e.g., two) securing pins 23 to pass therethrough and to engage the bone segment 3, respectively, thereby securing the guiding surgical instrument 2 on the bone segment 3.

Additionally, the guiding surgical instrument 2 is further formed with a guiding slot unit 24. The guiding slot unit 24 is disposed correspondingly with the operation route, and includes a plurality of guiding slots 241 so as to guide a cutting tool (not depicted in the drawings) to cut a portion of the bone segment 3 (e.g., the portion involving depressed fractures of the tibial plateau) along the operation route.

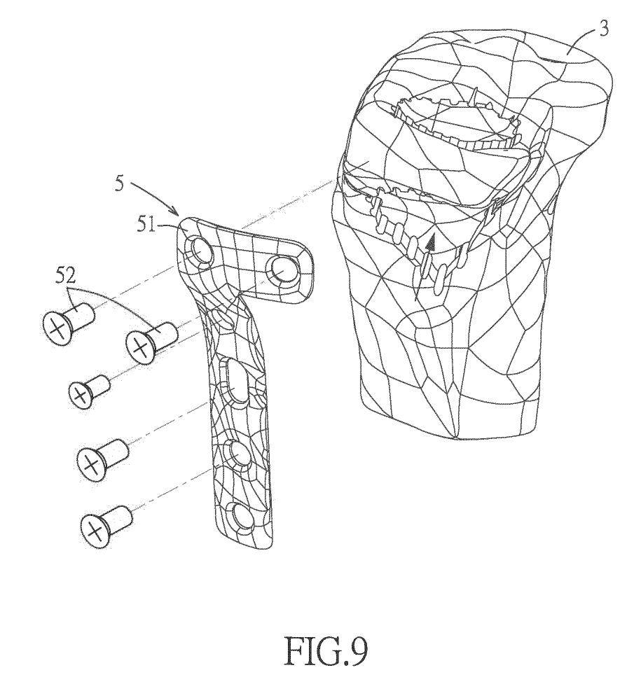

As shown in FIG. 9, after the cutting of the portion is completed, everything engaging the bone segment 3 (including the positioning components 122, the guiding surgical instrument 2 and the securing pins 23) may be removed from the bone segment 3, allowing operations that involve moving the portion of the bone segment 3 thus cut to be performed. In this embodiment, the portion that is cut is to be moved along a direction as depicted by the arrow in FIG. 9, in order to elevate the portion to an appropriate height for addressing the tibial plateau fracture with depression.

In step S06, the processor generates third geometric information regarding a shape of a second implant 5 based on the three-dimensional bone segment model. In some embodiments, after the portion of the bone segment 3 has been cut and moved to an appropriate position, imaging information associated with the bone segment 3 may be re-obtained to create a new three-dimension bone segment model which represents the bone segment 3 after the relocation of the portion thereof, and the third geometric information is generated based on the new three-dimension bone segment model.

It is noted that, with the third geometric information, the second implant 5 may be created physically using, for example, additive manufacturing or 3D printing.

As shown in FIG. 9, the second implant 5 is to be disposed on the bone segment 3 after the portion of the bone segment 3 has been cut and moved to the appropriate position, and after the first implant 4 and the guiding surgical instrument 2 have been removed from the bone segment 3, so as to secure the portion of the bone segment 3 at the appropriate position.

The second implant 5 includes a second bone plate 51, and a plurality of second bone screws 52 to be put into the bone segment 3 respectively at second spots thereof so as to secure the second bone plate 51 on the bone segment 3. It is noted that the third geometric information regarding the shape of the second implant 5 may be generated further based on the three-dimensional first implant model, so that the second spots of the bone segment 3 may be selected to be different from the first spots, at which the first bone screws 42 were put into the bone segment 3 for securing the first implant 4 on the bone segment 3. In this manner, the second implant 5 may be secured on the bone segment 3 with more stability.

Using the above method, the three-dimensional bone segment model and the three-dimensional first implant model may be created before an orthopedic surgery is performed, and the positioning surgical instrument 1 and the guiding surgical instrument 2 which serve as the patient specific instruments may be created accordingly. This may be helpful particularly in the cases where the bone segment 3 of the subject is already attached with an implant.

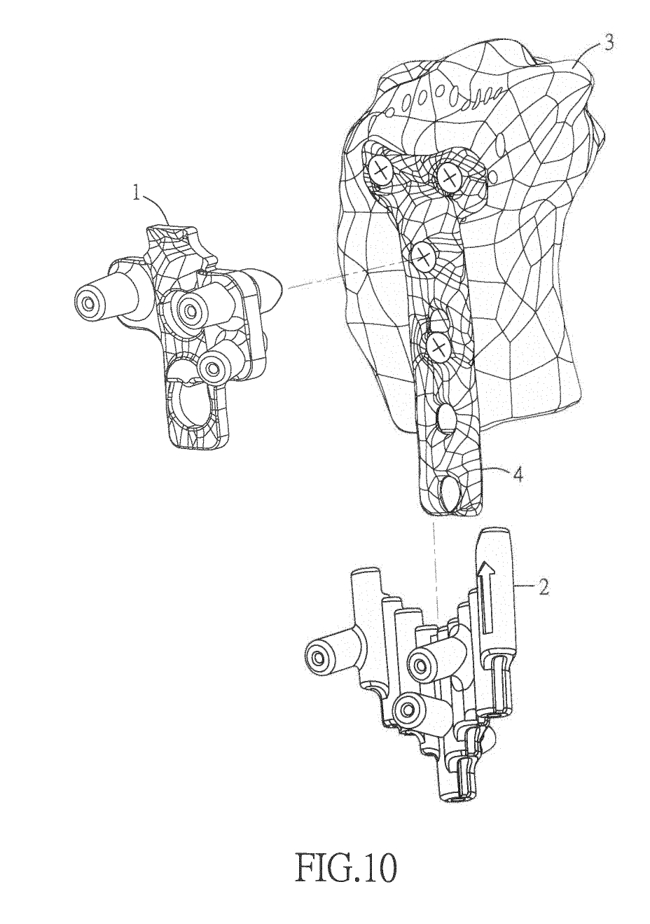

FIG. 10 illustrates an exemplary positioning surgical instrument 1 and an exemplary guiding surgical instrument 2 according to one embodiment of the disclosure. In this embodiment, the subject has a hybrid knee joint issue where the subject has a tibial plateau fracture with depression and a tilted tibial plateau. As a result, due to the operations that are needed to be performed, the positioning surgical instrument 1 and the guiding surgical instrument 2 may be shaped differently from those of the previous embodiment.

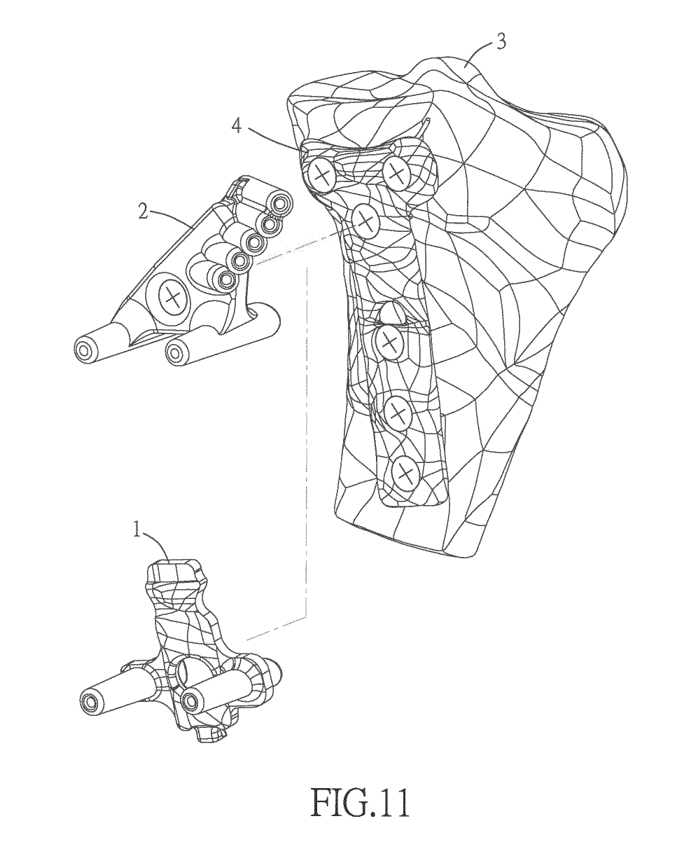

FIG. 11 illustrates an exemplary positioning surgical instrument 1 and an exemplary guiding surgical instrument 2 according to one embodiment of the disclosure. In this embodiment, knee joint issue of a tilted tibial plateau is diagnosed. As a result, due to the operations that are needed to performed, the positioning surgical instrument 1 and the guiding surgical instrument 2 may be shaped differently from those of the previous embodiments.

To sum up, the positioning surgical instrument 1 that is created using the above method allows the positioning component(s) 122 to pass therethrough to engage the bone segment 3, and the guiding surgical instrument 2 may be disposed on a predetermined location of the bone segment 3 using the positioning component(s) 122. In this manner, the guiding slot unit 24 may be disposed at the operation route to allow the cutting tool to cut the portion of the bone segment 3 along the operation route.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding various inventive aspects.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for designing a patient specific instrument for an orthopedic surgery, the method being implemented using a processor and comprising:
obtaining imaging information associated with a bone segment of a subject and a first implant fastened to a part of the bone segment;
creating a three-dimensional bone segment model for the bone segment and a three-dimensional first implant model for the first implant based on the imaging information associated with the bone segment and the first implant; and
generating first geometric information regarding a shape of a positioning surgical instrument based on the three-dimensional bone segment model and the three-dimensional first implant model, the positioning surgical instrument to be disposed on the bone segment and the first implant, the positioning surgical instrument being formed with at least one first positioning path that guides passage of a positioning component therethrough and aligns the positioning component with a positioning location of the bone segment which is not covered by the first implant and at which the positioning component is to engage the bone segment.

2. The method of claim 1, further comprising, prior to the generating first geometric information:
receiving a user-input operation route with respect to the three-dimensional bone segment model indicating at least one position of a cut to be performed on the bone segment during surgery;
wherein the generating first geometric information includes generating the first geometric information based on the three-dimensional bone segment model, the three-dimensional first implant model and the operation route, such that the first positioning path is formed on the positioning surgical instrument to allow the positioning component to engage the bone segment at the positioning location which corresponds to the position of the cut indicated by the operation route.

3. The method of claim 2, further comprising, after the creating a three-dimensional bone segment model and a three-dimensional first implant model:
generating second geometric information regarding a shape of a guiding surgical instrument based on the three-dimensional bone segment model and the user-input operation route, the guiding surgical instrument to be disposed on the bone segment with the first implant removed from the bone segment, the guiding surgical instrument being formed with at least one second positioning path configured for insertion by the positioning component which has engaged the bone segment at the positioning location, and being further formed with a guiding slot unit that is disposed correspondingly with the operation route, and that guides a cutting tool to cut a portion of the bone segment along the operation route.

4. The method of claim 3, further comprising:
generating third geometric information regarding a shape of a second implant based on at least the three-dimensional bone segment model, the second implant to be disposed on the bone segment after the portion of the bone segment has been cut, and after the first implant and the guiding surgical instrument have been removed from the bone segment.

5. The method of claim 4, the first implant including a first bone plate and a plurality of first bone screws to be put into the bone segment respectively at first spots thereof so as to secure the first bone plate on the bone segment, wherein:
the generation of the third geometric information regarding the shape of the second implant is further based on the three-dimensional first implant model; and
the second implant includes a second bone plate and a plurality of second bone screws to be put into the bone segment respectively at second spots thereof so as to secure the second bone plate on the bone segment, the first spots being different from the second spots.

6. The method of claim 1, wherein in the generating first geometric information, the positioning surgical instrument is further formed with a securing path that guides passage of a securing component therethrough and aligns the securing component with a securing location of the bone segment which is not covered by the first implant and at which the securing component is to be secured to the bone segment.

7. The method of claim 1, wherein:
the imaging information obtained by the processor includes a plurality of computed tomography (CT) scan images of the bone segment and the first implant;
the creating a three-dimensional bone segment model and a three-dimensional first implant model includes
processing the imaging information using one of interpolate correction, iterative correction and combines correction,
calculating contours of the bone segment and the first implant in each of the CT scan images based on the imaging information thus processed using region growing by pixel aggregation, and
creating the three-dimensional bone segment model and the three-dimensional first implant model based on the contours of the bone segment and the first implant in each of the CT scan images, respectively, by using the marching cubes algorithm.

8. A patient specific instrument for an orthopedic surgery, comprising:
a base shaped to be disposed on a bone segment of a subject and an implant fastened to a part of the bone segment; and
a plurality of positioning parts extending from said base, each of said positioning parts being formed with a first positioning slot that defines a first positioning path that guides passage of a positioning component therethrough, and aligns the positioning component with a positioning location of the bone segment which is not covered by the implant and at which the positioning component is to engage the bone segment.

9. The instrument of claim 8, further comprising at least one securing part extending from said base and formed with a securing slot,
the securing slot defining a securing path that guides passage of a securing component therethrough, and aligning the securing component with a securing location of the bone segment which is not covered by the implant and at which the securing component is to engage the bone segment.
